(19) United States
(12) Patent Application Publication
(10) Pub. No.: US 2024/0172984 A1
(43) Pub. Date: May 30, 2024

(54) ELECTROENCEPHALOGRAM (EEG) EMOTION RECOGNITION METHOD BASED ON SPIKING CONVOLUTIONAL NEURAL NETWORK

(71) Applicant: Hangzhou Dianzi University, Hangzhou City (CN)

(72) Inventors: Hong ZENG, Hangzhou City, Zhejiang Province (CN); Shuping LE, Hangzhou City, Zhejiang Province (CN); Feifan XU, Hangzhou City, Zhejiang Province (CN); Deng PAN, Hangzhou City, Zhejiang Province (CN); Haohao ZHENG, Hangzhou City, Zhejiang Province (CN); Yu OUYANG, Hangzhou City, Zhejiang Province (CN); Zhe JIA, Hangzhou City, Zhejiang Province (CN); Hongwei WANG, Hangzhou City, Zhejiang Province (CN)

(73) Assignee: Hangzhou Dianzi University, Hangzhou City (CN)

(21) Appl. No.: 18/510,727

(22) Filed: Nov. 16, 2023

(30) Foreign Application Priority Data
Nov. 28, 2022 (CN) 202211503605.3

(51) Int. Cl. A61B 5/374; A61B 5/00; A61B 5/16; G16H 10/20; G16H 50/20

(52) U.S. Cl. CPC A61B 5/374; A61B 5/165; A61B 5/7257; A61B 5/7267; G16H 10/20; G16H 50/20

(57) ABSTRACT

The present disclosure discloses an electroencephalogram (EEG) emotion recognition method based on a spiking convolutional neural network. In the present disclosure, after EEG emotion data is preprocessed, a differential entropy feature is extracted and a time window is intercepted, and then a spiking convolutional encoder is configured to encode the EEG emotion data; a spiking convolutional feature extractor extracts a spiking data feature; and a spiking fully connected classifier performs learning and classification. In the present disclosure, a spiking neural network combining a spiking convolutional layer and a spiking fully connected layer is trained directly to achieve an objective of EEG emotion classification. In a training process, spatio-temporal information in EEG data is extracted by transferring a spiking between layers of the network and between time slices by using the spiking convolutional layer, and then an emotion classification task is performed through feature learning of the spiking fully connected layer.

### CROSS REFERENCE TO RELATED APPLICATION

This patent application claims the benefit and priority of Chinese Patent Application No. 202211503605.3. filed with the China National Intellectual Property Administration on Nov. 28, 2022, the disclosure of which is incorporated by reference herein in its entirety as part of the present application.

### TECHNICAL FIELD

The present disclosure relates to the field of EEG emotion recognition technologies in the field of biometrics, and mainly relates to an electroencephalogram (EEG) emotion recognition method based on a spiking convolutional neural network.

### BACKGROUND

EEG data is data obtained by amplifying and recording a spontaneous biological potential of the brain from the scalp through a precision electronic device. A large amount of information about brain neuron activity is recorded in the EEG data, and the information includes rich spatio-temporal information, which can be used to obtain emotions of subjects. In recent years. more researches focus on emotion recognition, which provides a bridge for human-computer interaction.

A spiking neural network (SNN) includes neuron nodes with temporal dynamics characteristics and a low power consumption binary spiking transmission mode, which highly uses biological brain physical characteristics and a learning mode thereof for reference. Therefore, the spiking neural network has capabilities such as powerful spatio-temporal information representation, asynchronous event information processing, and low power consumption learning. In addition, cross fusion of the spiking neural network and a current computer science-oriented artificial neural network represented by a deep convolution network is considered as a powerful way to develop artificial general intelligence. In the face of the decreasing improvement of the deep CNN and the questioning of underlying rationality, the fusion and complementary development of the spiking neural network and the deep artificial neural network are gradually becoming the trend of the next generation of artificial neural network in the future.

The EEG data includes rich spatial information brought by different acquisition points and continuous temporal information brought by high-frequency sampling within a time period. On one hand, using a conventional artificial neural network EEG data processing and classification method such as CNN and a graph convolutional neural network (GCN) to extract EEG spatial information, and then using a method such as recurrent neural network (RNN) and a Long short-term memory network (LSTM) to extract temporal information for classification have achieved good results in emotion recognition accuracy. However, the high costs of multiplication and an excessively large model volume make it difficult to be widely used in portable embedded systems. On the other hand. although an existing spiking neural network model has good performance in energy consumption and biological interpretability, there are currently no breakthroughs in a standard method and accuracy and there are significant disadvantages in accuracy of EEG emotion classification.

Based on the above technical characteristics and comparing with an existing recognition method of the conventional artificial neural network, in the present disclosure, the spiking neural network is fused with an existing CNN, so that the network reaches a balance between performance and a volume, various kinds of EEG emotion data are effectively trained and recognized, and the network can be mounted in more efficient and portable neuromorphic hardware and embedded devices.

### SUMMARY

Aiming at disadvantages in the related art, the present disclosure provides an EEG emotion recognition method based on a spiking convolutional neural network. In the present disclosure, an existing conventional artificial neural network-based EEG emotion recognition method and a spiking neural network are combined. A spiking neural network combining a spiking convolutional layer and a spiking fully connected layer is directly trained to achieve an objective of EEG emotion classification. In a training process, spatio-temporal information in EEG data is extracted by transferring a spiking between layers of the network and between time slices by the spiking convolutional layer, and then an emotion classification task is performed through feature learning of the spiking fully connected layer. Finally, by using the method, running efficiency is ensured, and accuracy of EEG emotion classification is higher than that of the existing spiking neural network, which is approximate to that of the existing conventional artificial neural network, to achieve a balance between the running efficiency and performance.

To achieve the above objective. the technical solution in the present disclosure includes the following steps:

step 1: data set acquisition: acquiring EEG data of a subject when the subject watches movie clips, and immediately completing, by the subject, a questionnaire after watching each movie clip, to report an emotional response of the subject to each movie clip, where the emotional response includes positive, neutral, and negative; and the EEG data is EEG signals that are acquired by a 10-20 EEG system and that are at electrode positions designated by 32 channels;

step 2: data preprocessing: performing downsampling and ocular artifact removal preprocessing on an original EEG signal, filtering a time domain signal by using a Hanning window and performing fast Fourier transformation, performing window sliding by using a channel signal of each acquired electrode, and calculating differential entropy (Differential Entropy, DE) features of 32 channels at four frequency bands of all acquired electrodes;

step 3: sample generation: performing non-overlapping window sliding on processed differential entropy features at T time windows, and performing the operation on each channel at each frequency band, to obtain time*channel*frequency band; converting a one-dimensional channel data sequence into a two-dimensional mesh matrix sequence, where a position correspondence is obtained through a two-dimensional topology map of an EEG electrode cap; and finally obtaining an input sample: time*H*W*frequency band, where H and W are a height and a width of the two-dimensional mesh matrix sequence;

step 4: defining an input and an output of a model, where an input of single training of the model includes a batch of samples, and a structure of each sample is time*H*W*frequency band, and thus an input structure Input of the model is batch*time*H*W*frequency band; and an output of the model is a batch of vectors in a form of one-hot, and an output structure Output=batch*classes, where classes represent a probability that the sample belongs to the class, which is represented by using a decimal 0 to 1, and batch represents a quantity of batches;

step 5: defining a spiking neuron, where

the spiking neuron controls generation and propagation of a signal in the network as a basic unit of the spiking neural network, and in the present disclosure, the used neuron model is a Leaky integrity Fire (LIF) model, and an available model dynamics equation is as follows:

$${H}_{t} = ( 1 - \frac{1}{c} ) {V}_{t - 1} + \frac{1}{c} {X}_{t} and {X}_{t} = wI ( t ) \qquad ( 1 )$$

where in the formula (1), Hₜ and Vₜ respectively represent a membrane potential after dynamical change of a neuron and a membrane potential after a spiking is triggered at a time step; Xₜ represents an external input when V₁₋₁=0; an integration progress

$$\frac{1}{c} {X}_{t}$$

enables an LIF neuron to remember current input information, and

$$( 1 - \frac{1}{c} ) {V}_{t - 1}$$

may be considered as a forgetfulness of information from the past; and the formula indicates that a balance between memory and forgetting is controlled by a membrane time constant c=1.2;

$${S}_{t} = Θ H ( {H}_{{t}^{-}} {V}_{t h} ) \qquad ( 2 )$$

$$and$$

$$Θ ( x ) = \{ \begin{matrix}1 , x \geq 0 \\ 0 , x < 0\end{matrix}$$

where in the formula (2), Sₜ represents an output spiking at time t, which describes a spiking generation process, and Θ(x) is a Heaviside step function, which is defined as follows: Θ(x)=1 when x≥0 and Θ(x)=0 when x<0;

Vₜ=Hₜ(1−Sₜ)+VᵣₑₛₑₜSₜ (3)

where the formula (3) describes a process of the membrane potential returns to Vreset after the spiking is generated and is of a hard reset type, which is applicable to a deep spiking neural network of the model; and

the model is a spiking neural network combining a conventional CNN, a fully connected network, and a spiking neuron mechanism, and the model performs, by using a modified activation function and an activation function of an activation model of the spiking neuron and a surrogate gradient function, input and output between layers in the activation mode in a network training process, and also sequentially performs spiking output, activation, and transmission on an intra-layer time slice;

step 6: defining a spiking neural network architecture, where

a spiking neural network structure includes three parts of an adaptive spiking convolutional encoder, a spiking convolutional feature extraction network, and a spiking fully connected classifier;

the adaptive spiking convolutional encoder is used as the topmost layer of the spiking neural network structure and aims at self-learning real-valued sample data into a binary output in a spiking convolution manner in a spiking convolution manner, and subsequently, the spiking neural network structure performs processing and classification by using a spiking value as data;

the spiking convolutional feature extraction network learns and extracts effective temporal information and spatial information in EEG sample data through output interaction between layers of a multi-layer spiking convolutional neural network and intra-layer temporal slice sequential interaction, so that extracted spiking feature information can be effectively used for emotion classification and recognition; and

the spiking fully connected classifier obtains a final spiking neuron weight through output interaction between layers of a two-layer spiking fully connected network and intra-layer temporal slice sequential interaction learning and then obtains a last layer of spiking output. Herein, due to a characteristic of spiking output binarization, generality and robustness of the model are greatly reduced by directly using the spiking output as a classification result. Therefore, in the present disclosure, a final output of the spiking fully connected layer is ten times of classification classes, and then voting processing of ten to one is performed by using an average pooling operation to finally obtain a classification class;

step 7: defining a target function, where the target function used when the spiking neural network is trained is a mean square error function, in the mean square error function, x represents an input data sample, y represents an output label generated during training of the model, and i represents each value in a sample matrix, and a formula (4) of the mean square error function is as follows:

MSE(xᵢ,yᵢ)=(xᵢ−yᵢ)² (4); and

step 8: training and test: inputting a training set into the spiking neural network according to the input structure defined in step 3 for a plurality of rounds of training; and inputting a test set used for a trained model into the network in a same manner for prediction after each round of training, comparing a predicted result that is used as an output with a real label to calculate a mean square error, and finally obtaining overall classification accuracy of the spiking neural network on the test set.

Compared with the related art, the present disclosure has the following beneficial effects:

The present disclosure provides an EEG emotion recognition method based on a spiking convolutional neural network. A spiking neuron mechanism is effectively combined with a conventional CNN and a fully connected neural network, time domain information of EEG data is more effectively extracted. A strong generalization capability of the model is applicable to a plurality of data sets. Emotion recognition accuracy is improved compared with that in a conventional spiking neural network with a same structure. Compared with the conventional neural network with the same structure, the model has a great reduction in a running speed and a volume, and has wide application prospects in neuromorphic hardware and portable embedded devices.

### BRIEF DESCRIPTION OF THE DRAWINGS

To describe the embodiments of the present disclosure and the technical solutions more clearly, the following briefly describes the accompanying drawings required for describing the examples or the prior art. Apparently, the accompanying drawings in the following description merely show some examples of the present disclosure, and those of ordinary skill in the art may still derive other accompanying drawings from these accompanying drawings without creative efforts.

FIG. 1 is a flowchart of an EEG emotion classification method based on a spiking neural network according to the present disclosure;

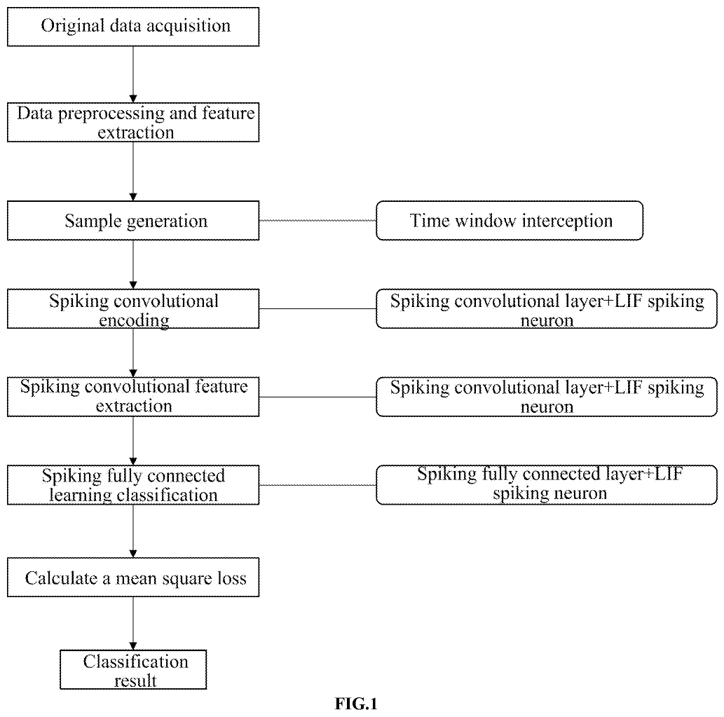

FIG. 2 is a schematic diagram of structure definition and internal neurodynamic of an LIF neuron model according to the present disclosure;

FIG. 3 is a schematic diagram of an EEG emotion classification network structure based on a spiking neural network according to the present disclosure; and

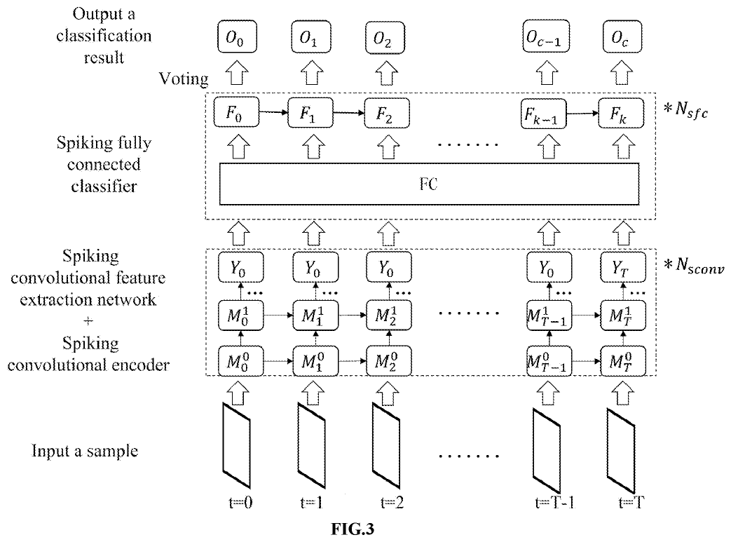

FIG. 4 is an experimental performance comparison between an EEG emotion classification method based on a spiking neural network and another existing spiking neural network method according to the present disclosure.

### DETAILED DESCRIPTION OF THE EMBODIMENTS

The following describes present disclosure more clearly and completely with reference to the accompanying drawings and the embodiments, so that the advantages and features of the present disclosure can be more easily understood by a person skilled in the art. All other embodiments obtained by those skilled in the art based on the embodiments of the present disclosure without creative efforts shall fall within the protection scope of the present disclosure.

### Step 1. Acquisition of Original Data.

First, a subject wears a 32-electrode EEG acquisition headgear designed based on an international 10-20 standard, 15 movie clips (which include a positive emotion, a neutral emotion, and a negative emotion) are selected from a video material library as emotion stimulus sources used in the experiment. The subject watches 15 clips in each experiment, with a five-second prompt before each clip. Each clip is presented for four minutes, and self-assessment is performed for 45 seconds after each clip ends. At the self-assessment stage, a questionnaire is completed to feed back an emotion response of the subject to each movie clip. Data is reduced to 200 Hz by using an EEGLAB tool to remove signal interference of ophthalmic and myoelectric signals, a band-pass frequency filter of 0 to 7 Hz is applied to filter, and then an emotion label is marked on corresponding data (−1 represents negative, 0 represents neutral, and +1 represents positive), and finally the data is stored in a file in a mat format.

### Step 2. Data Preprocessing and Feature Extraction.

A DE feature is extracted for each channel at four frequency bands: θ band (4 Hz to 7 Hz), α band (8 Hz to 13 Hz), β band (14 Hz to 30 Hz), and γ band (31 Hz to 50 Hz). A specific method is as follows.

Original data is filtered by using a non-overlapping sliding window; fast Fourier transformation is performed on the data every one second, and differential entropies of the four frequency bands are calculated. The differential entropy is defined as follows.

The differential entropy DE is a generalization form of a Shannon information entropy −Σₓap(x)log(p(x)) in a continuous variable entropy.

DE=−∫ₐ^{b}p(x)log(p(x)dx (1)

where p(x) represents a probability density function of continuous information, [a, b] represents a value interval of EEG data. For EEG data of a specific length that approximately obeys Gaussian distribution N(μ, σ²), a differential entropy is:

$$DE = - {\int }_{a}^{b} \frac{1}{\sqrt{2 {πσ}_{i}^{2}}} {e}^{\frac{{( x - μ )}^{2}}{2 {σ}_{i}^{2}}} log ( \frac{1}{\sqrt{2 {πσ}_{i}^{2}}} {e}^{\frac{{( x - μ )}^{2}}{2 {σ}_{i}^{2}}} ) dx = \frac{1}{2} log ( 2 π e {σ}_{i}^{2} ) \qquad ( 2 )$$

z-score normalization is performed on processed DE EEG data, where a normalization formula (3) is as follows:

$$U = \frac{X - \overline{X}}{S} \qquad ( 3 )$$

where X is an EEG signal on each channel, X̅ is an average value of the EEG signal on each channel, and S is a standard deviation of the EEG signal on each channel.

### Step 3. Sample Generation.

Non-overlapping window sliding is performed on the processed 0.5 s window DE features at T 32 time windows, and the operation is performed on 32 channels at four frequency bands, to obtain 32*32*4 (time*channel*frequency band); and then a one-dimensional channel data sequence is converted into a 9*9 two-dimensional mesh matrix sequence. A finally obtained input sample is 32*9*9*4 (time*H*W*frequency band).

### Step 4. Define an Input and an Output of a Spiking Neural Network Model.

An input of single training of the spiking neural network model includes a batch of 16 samples. A structure of each sample is a structure (32*9*9*4) of the input sample given in step 3. Therefore, an input structure of the spiking neural network Input=16*32*9*9*4. An output structure of the spiking neural network is a batch of 16 vectors in a form of one-hot, and a structure Output=batch*classes, where classes represent a probability that the sample belongs to the class, which is represented by using a decimal 0 to 1.

### Step 5. Define a Spiking Neuron.

The spiking neuron controls generation and propagation of a signal in the network as a basic unit of the spiking neural network, and in the present disclosure, the used neuron model is an LIF model, and an available model dynamics equation is as follows:

Hₜ=(1−1/c)Vₜ₋₁+1/c Xₜ (4) and

Xₜ=wI(t)

where in the formula (4), Hₜ and Vₜ respectively represent a membrane potential after dynamical change of a neuron and a membrane potential after a spiking is triggered at a time step. Xₜ represents an external input when Vₜ₋₁=0. An integration progress

$$\frac{1}{c} {X}_{t}$$

enables an LIF neuron to remember current input information, and

$$( 1 - \frac{1}{c} ) {V}_{t - 1}$$

may be considered as a forgetfulness of information from the past. The formula indicates that a balance between memory and forgetting is controlled by a membrane time constant c=1.2. In the model, a membrane time constant spiking convolutional layer c=1.2, and a spiking fully connected layer c=1.2.

$${S}_{t} = Θ ( {H}_{{t}^{-}} {V}_{t h} ) \qquad ( 5 )$$

$$and$$

$$Θ ( x ) = \{ \begin{matrix}1 , x \geq 0 \\ 0 , x < 0\end{matrix}$$

where in the formula (5), Sₜ represents an output spiking at time t, which describes a spiking generation process, and Θ(x) is a Heaviside step function, which is defined as follows: Θ(x)=1 when x≥0 and Θ(x)=0 when x<0.

Vₜ=Hₜ(1−Sₜ)+VᵣₑₛₑₜSₜ (6)

where the formula (6) describes a process of the membrane potential returns to Vreset after the spiking is generated and is of a hard reset type, which is applicable to a deep spiking neural network of the model.

To resolve a spiking neuron output, that is, in the formula (5), according to the definition, a derivative is an impulse function:

$${Θ}^{′} ( x ) = \{ \begin{matrix}+ ∞ , x = 0 \\ 0 , x \neq 0\end{matrix} \qquad ( 7 )$$

Direct use of the impulse function for gradient descent obviously causes network training to be extremely unstable, so that in the present disclosure, a surrogate gradient function commonly used in the spiking neural network for direct training is introduced, and a principle is that y=Θ(x) is used in forward propagation and \dot{y}′=σ′(x), σ(x) is used as a surrogate function in backward propagation, and the surrogate gradient function is a similar function with a shape approaching Θ(x), but a smooth continuous function. In the present disclosure, the used surrogate function is a Surrogate arctanx function, which is defined as follows:

σ(x, α)=arctan(αx) (8)

where a parameter α is an adjustable parameter and is used for controlling a gradient, and α is set to 2.0.

### Step 6. Define a Spiking Neural Network Model Architecture.

An adaptive spiking convolutional encoder is used as a top layer of the model to receive input data, and then the input data is output to a spiking neural network model main body, where the model main body includes a spiking convolutional feature extraction network and a spiking fully connected classifier and implements extraction and classification of a temporal feature and a spatial feature of EEG emotion data, and a specific implementation includes the following steps.

Step 6-1. Define the adaptive spiking convolutional encoder as the topmost layer of the network, where the adaptive spiking convolutional encoder aims at self-learning real-valued sample data into a binary output in an appropriate spiking form in a spiking convolution manner, and a structure of the adaptive spiking convolutional encoder includes:

(1) a spiking convolutional layer 1, where a parameter of the spiking convolutional layer 1 is that a quantity of input channels is 4; a quantity of output channels is 64; kernelsize=(3, 3); padding=1; and bias=False;

(2) BatchNorm2d, where normalization is based on a formula (9) as follows:

$$y = \frac{x - mean ( x )}{\sqrt{Var ( x )} + eps} * gamma + beta \qquad ( 9 )$$

where eps is a value added to a denominator for stability of calculation and is 1e⁻⁵ by default;

gamma and beta are normalization coefficients, are not added by default, and are 1 and 0; and

mean(x) and Var(x) are a mean value and a variance of normalized data; and

(3) an LIF neuron activation layer, where a parameter of the LIF neuron activation layer is that a membrane time constant=1.2; and a surrogate gradient function=Surrogatearctanx.

Step 6-2. Define the spiking convolutional feature extraction network, where the spiking convolutional feature extraction network learns and extracts effective temporal information and spatial information in EEG sample data through output interaction between layers of a multi-layer spiking convolutional neural network and intra-layer temporal slice sequential interaction. A structure of the spiking convolution feature extraction network includes:

(1) a spiking convolutional layer 2, where a parameter of the spiking convolutional layer 2 is that a quantity of input channels is 64; a quantity of output channels is 128; kernelsize=(3,3); padding=1; and bias=False;

BatchNorm2d; and

an LIF neuron activation layer, where a parameter of the LIF neuron activation layer is that a membrane time constant=1.2; and a surrogate gradient function=Surrogatearctanx;

(2) a spiking convolutional layer 3, where a parameter of the spiking convolutional layer 3 is that a quantity of input channels is 128; a quantity of output channels is 256; kernelsize=(3,3); padding=1; and bias=False;

BatchNorm2d; and

an LIF neuron activation layer, where a parameter of the LIF neuron activation layer is that a membrane time constant=1.2; and a surrogate gradient function=Surrogatearctanx; and

(3) a spiking convolutional layer 4, where a parameter of the spiking convolutional layer 4 is that a quantity of input channels is 256; a quantity of output channels is 64; kernelsize=(1,1); and bias=False;

BatchNorm2d; and

an LIF neuron activation layer, where a parameter of the LIF neuron activation layer is that a membrane time constant=1.2; and a surrogate gradient function=Surrogatearctanx.

Step 6-3. Define the spiking fully connected classifier, where the spiking fully connected classifier obtains a final spiking neuron weight through output interaction between layers of a two-layer spiking fully connected network and intra-layer temporal slice sequential interaction learning, and finally obtains a classified output is finally obtained. A structure of the spiking fully connected classifier includes two spiking fully connected layers and one average pooled voting layer, and the structure of the spiking fully connected classifier is as follows:

(1) Dropout, where a parameter thereof is that p=0.25;

a spiking fully connected layer 1, where a parameter of the spiking fully connected layer 1 is that a quantity of input channels is 64*9*9; a quantity of output channels is 64*6*6; and bias=False; and

an LIF neuron activation layer, where a parameter of the LIF neuron activation layer is that a membrane time constant=2.0; and a surrogate gradient function=Surrogatearctanx;

(2) Dropout, where a parameter thereof is that p=0.25;

a spiking fully connected layer 2, where a parameter of the spiking fully connected layer 2 is that a quantity of input channels is 64*6*6; a quantity of output channels is 20; and bias=False; and

an LIF neuron activation layer, where a parameter of the LIF neuron activation layer is that a membrane time constant=2.0; and a surrogate gradient function=Surrogatearctanx; and

(3) an average pooled voting layer, where a parameter of the average pooled voting layer is that a voting ratio=10.

Step 7. Define a target function, where the target function used when the spiking neural network is trained is a mean square error function (MSE Loss), in the mean square error function, x represents an input data sample, y represents an output label generated during training of the model, and i represents each value in a sample matrix, and a formula (10) of the mean square error function is as follows:

MSE(xᵢ,yᵢ)=(xᵢ−yᵢ)² (10)

### Step 8. Model Training.

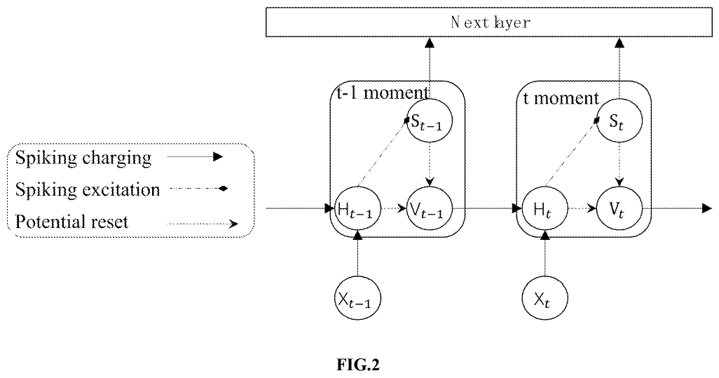

For the spiking convolutional layer and the spiking fully connected layer in step 6, a training process of a batch of inputs (16*32*9*9*4) may be summarized as follows: first, the inputs (16*32*9*9*4) are transposed into (32*16*9*9*4), that is, (T*batch*H*W*frequency band), for time step simulation of the spiking neural network, and a whole batch of data may be calculated to improve efficiency. Then, the spiking time step simulation during training is shown in FIG. 2. A left i−1 node is recorded as a state when an LIF neuron node is at i−1, and a right i node represents a state when the LIF neuron node is at i. A membrane potential Hₜ of a right neuron is charged from left Vₜ₋₁. When Hₜ₋₁ obtains an input Xₜ₋₁ at t−1, Hₜ₋₁ changes according to the formula (1), and if a potential is greater than a threshold, a spiking Sₜ₋₁ is generated. Hₜ₋₁ is reset to a triggered membrane potential Vₜ₋₁ according to the formula (3), and if no spiking is generated, Vₜ₋₁ may be directly obtained. Then, the generated spiking becomes an input of a next layer, that is, the triggered membrane potential Vₜ₋₁ becomes a pre-activation potential Hₜ₋₁ of a next time step. The process is performed on all nodes of a layer in a time sequence, to achieve a function of learning and transferring temporal information between adjacent spiking neuron nodes.

### Step 9. Model Test and Result Feedback.

A trained model is obtained through the foregoing steps. An EEG test data set is input into the trained spiking neural network, to obtain an emotion classification result of the EEG data, and then an optimal result is selected from all test result in each round of test for storage. Then, three types of experimental data of each of 32 subjects are averaged to obtain final test accuracy.

### Step 10. Result Evaluation.

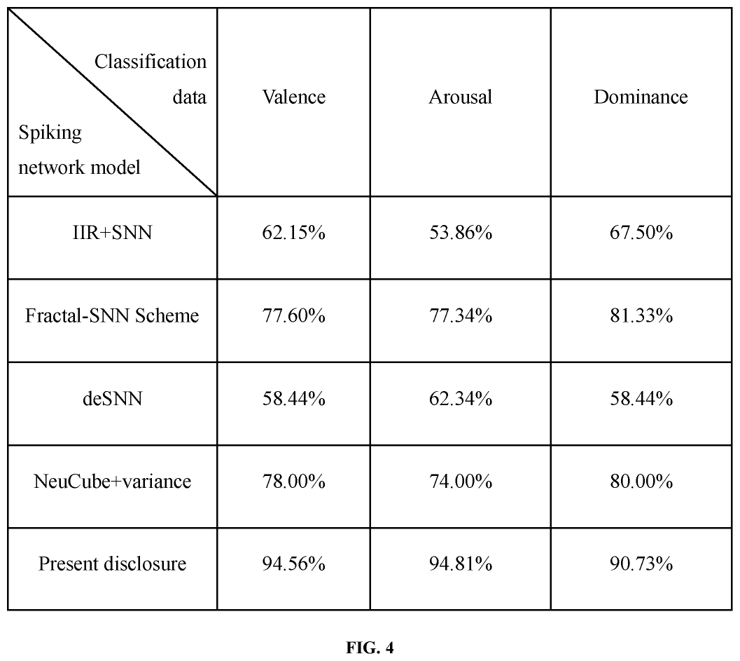

To verify effectiveness of the present disclosure, three types of emotional experimental data are selected from 32 subjects, which are respectively Valence (valence), Arousal (arousal), and Dominance (dominance). Binary classification of emotions is performed by using the spiking neural network, to evaluate a feature extraction effect of the model. Through experimental verification, as shown in FIG. 4, compared with another method, the accuracy of the method in the present disclosure respectively reaches 94.56%, 94.81% and 90.73%, which reflects that a better and stronger generalization effect on an image feature extraction in EEG data is achieved in the present disclosure, and the classification accuracy in emotion recognition is obviously higher than that of another existing spiking neural network method.

What is claimed is:

1. An electroencephalogram (EEG) emotion recognition method based on a spiking convolutional neural network, comprising the following steps:
step 1: data set acquisition: acquiring EEG data of a subject when the subject watches movie clips, and immediately completing, by the subject, a questionnaire after watching each movie clip, to report an emotional response of the subject to each movie clip, wherein the emotional response comprises positive, neutral, and negative; and the EEG data is EEG signals that are acquired by a 10-20 EEG system and that are at electrode positions designated by 32 channels;
step 2: data preprocessing: performing downsampling and ocular artifact removal preprocessing on an original EEG signal, filtering a time domain signal by using a Hanning window and performing fast Fourier transformation, performing window sliding by using a channel signal of each acquired electrode, and calculating differential entropy features of 32 channels at four frequency bands of all acquired electrodes;
step 3: sample generation: performing non-overlapping window sliding on processed differential entropy features at T time windows, and performing the operation on each channel at each frequency band, to obtain time*channel*frequency band; converting a one-dimensional channel data sequence into a two-dimensional mesh matrix sequence, wherein a position correspondence is obtained through a two-dimensional topology map of an EEG electrode cap; and finally obtaining an input sample: time*H*W*frequency band, wherein H and W are a height and a width of the two-dimensional mesh matrix sequence;
step 4: defining an input and an output of a model, wherein an input of single training of the model comprises a batch of samples, a structure of each sample is time*H*W*frequency band, and thus an input structure Input of the model is batch*time*H*W*frequency band; and an output of the model is a batch of vectors in a form of one-hot, and an output structure Output=batch*classes, wherein classes represent a probability that the sample belongs to the class, which is represented by using a decimal 0 to 1, and batch represents a quantity of batches;
step 5: defining a spiking neuron, wherein
a leaky-integrate-fire (LIF) model is used as a neuron model; and the LIF model is a spiking neural network combining a conventional CNN, a fully connected network, and a spiking neuron mechanism, and performs, by using a modified activation function and an activation function of an activation model of the spiking neuron with help of a surrogate gradient function, input and output between layers in the activation mode in a network training process, and also sequentially performs spiking output, activation, and transmission on an intra-layer time slice;
step 6: defining a spiking neural network architecture, wherein
a spiking neural network structure comprises an adaptive spiking convolutional encoder, a spiking convolutional feature extraction network, and a spiking fully connected classifier;
step 7: defining a target function, wherein the target function used when the spiking neural network is trained is a mean square error function; and
step 8: training and test: inputting a training set into the spiking neural network according to the input structure defined in step 3 for a plurality of rounds of training; and inputting a test set used for a trained model into the network in a same manner for prediction after each round of training, comparing a predicted result that is used as an output with a real label to calculate a mean square error, and finally obtaining overall classification accuracy of the spiking neural network on the test set.

2. The EEG emotion recognition method based on a spiking convolutional neural network according to claim 1, wherein the used neuron model is the LIF model, and an available model dynamics equation is as follows:
$${H}_{t} = ( 1 - \frac{1}{c} ) {V}_{t - 1} + \frac{1}{c} {X}_{t} \qquad ( 4 )$$
$$and$$
$${X}_{t} = wI ( t )$$
wherein in the formula (4), Hₜ and Vₜ respectively represent a membrane potential after dynamical change of a neuron and a membrane potential after a spiking is triggered at a time step; Xₜ represents an external input when Vₜ₋₁=0; an integration progress
$$\frac{1}{c} {X}_{t}$$
enables an LIF neuron to remember current input information, and
$$( 1 - \frac{1}{c} ) {V}_{t - 1}$$
is considered as a forgetfulness of information from the past; and the formula indicates that a balance between memory and forgetting is controlled by a membrane time constant c=1.2, and in the model, a membrane time constant spiking convolutional layer c=1.2, and a spiking fully connected layer c=1.2;
$${S}_{t} = Θ ( {H}_{{t}^{-}} {V}_{t h} ) \qquad ( 5 )$$
$$and$$
$$Θ ( x ) = \{ \begin{matrix}1 , x \geq 0 \\ 0 , x < 0\end{matrix}$$
wherein in the formula (5), Sₜ represents an output spiking at time t, which describes a spiking generation process, and Θ(x) is a Heaviside step function, which is defined as follows: Θ(x)=1 when x≥0 and Θ(x)=0 when x<0;
Vₜ=Hₜ(1−Sₜ)+VᵣₑₛₑₜSₜ (6)
wherein the formula (6) describes a process of the membrane potential returns to Vreset after the spiking is generated and is of a hard reset type;
to resolve output of the spiking neuron output, that is, in the formula (5), according to the definition, a derivative is an impulse function:
$${Θ}^{′} ( x ) = \{ \begin{matrix}+ ∞ , x = 0 \\ 0 , x \neq 0\end{matrix} \qquad ( 7 )$$
direct use of the impulse function for gradient descent obviously causes network training to be extremely unstable, so that a surrogate gradient function commonly used in the directly trained spiking neural network is introduced, y=Θ(x) is used in forward propagation and \dot{y}′=σ′(x), σ(x) is used as a surrogate function in backward propagation, the surrogate gradient function is a similar function with a shape approaching Θ(x), but a smooth continuous function, and the used surrogate function is a Surrogate arctanx function, which is defined as follows:
σ(x, α)=arctan(αx) (8)
wherein a parameter α is an adjustable parameter and is used for controlling a gradient, and α is set to 2.0.

3. The EEG emotion recognition method based on a spiking convolutional neural network according to claim 2, wherein the adaptive spiking convolutional encoder is used as a top layer of the model to receive input data, and then the input data is output to a spiking neural network model main body, wherein the model main body comprises the spiking convolutional feature extraction network and the spiking fully connected classifier and implements extraction and classification of a temporal feature and a spatial feature of EEG emotion data, and a specific implementation comprises:
step 6-1: defining the adaptive spiking convolutional encoder as the topmost layer of the network, wherein the adaptive spiking convolutional encoder aims at self-learning real-valued sample data into a binary output in an appropriate spiking form in a spiking convolution manner, and a structure of the adaptive spiking convolutional encoder comprises:
(1) a spiking convolutional layer 1, wherein a parameter of the spiking convolutional layer 1 is that a quantity of input channels is 4; a quantity of output channels is 64; kernelsize=(3,3); padding=1; and bias=False;
(2) BatchNorm2d, wherein normalization is based on a formula (9) as follows:
$$y = \frac{x - mean ( x )}{\sqrt{Var ( x )} + eps} * gamma + beta \qquad ( 9 )$$
wherein eps is a value added to a denominator for stability of calculation and is 1e⁻⁵ by default;
gamma and beta are normalization coefficients, are not added by default, and are 1 and 0; and
mean(x) and Var(x) are a mean value and a variance of normalized data; and
(3) an LIF neuron activation layer, wherein a parameter of the LIF neural activation layer is that a membrane time constant=1.2; and a surrogate gradient function=Surrogatearctanx;
step 6-2: defining the spiking convolutional feature extraction network, wherein the spiking convolutional feature extraction network learns and extracts effective temporal information and spatial information in EEG sample data through output interaction between layers of a multi-layer spiking convolutional neural network and intra-layer temporal slice sequential interaction; and a structure of the spiking convolutional neural network comprises:
(1) a spiking convolutional layer 2, wherein a parameter of the spiking convolutional layer 2 is that a quantity of input channels is 64; a quantity of output channels is 128; kernelsize=(3,3); padding=1; and bias=False;
BatchNorm2d; and
an LIF neuron activation layer, wherein a parameter of the LIF neuron activation layer is that a membrane time constant=1.2; and a surrogate gradient function=Surrogatearctanx;
(2) a spiking convolutional layer 3, wherein a parameter of the spiking convolutional layer 3 is that a quantity of input channels is 128; a quantity of output channels is 256; kernelsize=(3,3); padding=1; and bias=False; and
(3) a spiking convolutional layer 4, wherein a parameter of the spiking convolutional layer 4 is that a quantity of input channels is 256; a quantity of output channels is 64; kernelsize=(1,1); and bias=False; and
step 6-3: defining the spiking fully connected classifier, wherein the spiking fully connected classifier obtains a final spiking neuron weight through output interaction between layers of a two-layer spiking fully connected network and intra-layer temporal slice sequential interaction learning, and finally obtains a classified output; and a structure of the spiking fully connected classifier comprises two spiking fully connected layers and one average pooled voting layer, and the structure of the spiking fully connected classifier comprises:
(1) Dropout, wherein a parameter thereof is that p=0.25;
a spiking fully connected layer 1, wherein a parameter of the spiking fully connected layer 1 is that a quantity of input channels is 64*9*9; a quantity of output channels is 64*6*6; and bias=False; and
an LIF neuron activation layer, wherein a parameter of the LIF neuron activation layer is that a membrane time constant=2.0; and a surrogate gradient function=Surrogatearctanx;
(2) Dropout, wherein a parameter thereof is that p=0.25;
a spiking fully connected layer 2, wherein a parameter of the spiking fully connected layer 2 is that a quantity of input channels is 64*6*6; a quantity of output channels is 20; and bias=False; and
(3) an average pooled voting layer, wherein a parameter of the average pooled voting layer is that a voting ratio=10.

4. The EEG emotion recognition method based on a spiking convolutional neural network according to claim 2, wherein for the spiking convolutional layer and the spiking fully connected layer in step 6, a training process of a group of inputs is summarized as follows: first, the inputs 16*32*9*9*4 are transposed into 32*16*9*9*4 for time step simulation of the spiking neural network, and a whole batch of data is calculated to improve efficiency; and then the following spiking time step simulation is performed during training: a left i−1 node is recorded as a state when an LIF neuron node is at i−1, and a right i node represents a state when the LIF neuron is at i; a membrane potential Hₜ of a right neuron is charged from left Vₜ₋₁; when Hₜ₋₁ obtains an input Xₜ₋₁ at t−1, Hₜ₋₁ changes according to the formula (1), and if a potential is greater than a threshold, a spiking Sₜ₋₁ is generated; Hₜ₋₁ is reset to a triggered membrane potential Vₜ₋₁ according to the formula, and if no spiking is generated, Vₜ₋₁ is directly obtained; and then the generated spiking becomes an input of a next layer, that is, the triggered membrane potential Vₜ₋₁ becomes a pre-activation potential Hₜ₋₁ of a next time step, wherein the process is performed on all nodes of a layer in a time sequence, to achieve a function of learning and transferring temporal information between adjacent spiking neuron nodes.

5. The EEG emotion recognition method based on a spiking convolutional neural network according to claim 3, wherein for the spiking convolutional layer and the spiking fully connected layer in step 6, a training process of a group of inputs is summarized as follows: first, the inputs 16*32*9*9*4 are transposed into 32*16*9*9*4 for time step simulation of the spiking neural network, and a whole batch of data is calculated to improve efficiency; and then the following spiking time step simulation is performed during training: a left i−1 node is recorded as a state when an LIF neuron node is at i−1, and a right i node represents a state when the LIF neuron is at i; a membrane potential Hₜ of a right neuron is charged from left Vₜ₋₁; when Hₜ₋₁ obtains an input Xₜ₋₁ at t−1, Hₜ₋₁ changes according to the formula (1), and if a potential is greater than a threshold, a spiking Sₜ₋₁ is generated; Hₜ₋₁ is reset to a triggered membrane potential Vₜ₋₁ according to the formula, and if no spiking is generated, Vₜ₋₁ is directly obtained; and then the generated spiking becomes an input of a next layer, that is, the triggered membrane potential Vₜ₋₁ becomes a pre-activation potential Hₜ₋₁ of a next time step, wherein the process is performed on all nodes of a layer in a time sequence, to achieve a function of learning and transferring temporal information between adjacent spiking neuron nodes.
